(19) United States
(12) Patent Application Publication
(10) Pub. No.: US 2024/0398304 A1
(43) Pub. Date: Dec. 5, 2024

(54) APPARATUS AND METHOD FOR DETECTING AND RECOGNIZING HUMAN ACTIVITIES AND MEASURING ATTENTION LEVEL

(71) Applicant: AI Mnemonic Limited, Hong Kong (HK)

(72) Inventors: Wai Kit Ringo LAM, Hong Kong (HK); Wing Kuen LING, Hong Kong (HK); Cheuk Ho LEUNG, Hong Kong (HK); Hon Wing PANG, Hong Kong (HK); Luen Hon SIU, Hong Kong (HK); Yuk Fan HO, Hong Kong (HK)

(21) Appl. No.: 18/328,500

(22) Filed: Jun. 2, 2023

(51) Int. Cl. A61B 5/16; A61B 5/00; A61B 5/0205; A61B 5/11; A61B 5/369

(52) U.S. Cl. CPC A61B 5/168; A61B 5/0205; A61B 5/1118; A61B 5/369; A61B 5/4803; A61B 5/7203; A61B 5/02433; A61B 2503/12; A61B 2562/0219

(57) ABSTRACT

A method for recognizing a participating activity and computing an attention level of a subject from multi-model signals, comprising receiving the multi-model signals comprising an electroencephalogram (EEG) signal, a photoplethysmography (PPG) signal, an image/video signal, an audio signal, and an inertial measurement signal. The method further comprises executing an activity recognition to predict an activity type of a participating activity being performed by the subject using one or more of the multi-model signals; and executing an attention level computation to predict the subject's attention level in performing the participating activity using one or more of the multi-model signals.

### FIELD OF THE INVENTION

The present invention generally relates to techniques of detecting and recognizing human activities and measuring one's attention level. More specifically the present invention relates to methods and systems of using electroencephalogram (EEG), photoplethysmography (PPG), and other sensing mechanisms in detecting and recognizing human activities and measuring one's attention level.

### BACKGROUND OF THE INVENTION

Attention is crucial in education and workplace. It helps one to learn in school, improve work efficiency, and prevent mistakes and accidents. The degree of attention can be directly measured on a subject's brain activity or indirectly through the subject's behavior. Currently, direct measurement of brain activity is generally implemented using EEG, and the indirect methods may use computer vision for eye tracking, facial features, and pose estimation to calculate the attention level. Although the EEG is currently the best method to quantitatively measure attention level, it has a number of shortcomings. For example, EEG measurement is susceptible to poor electrode contact of the scalp and the skin. Besides, a high attention level indicated by EEG measurements does not necessarily mean the subject is actually focusing on the participating activity; the subject may be paying high attention to other unconcerned activities during the EEG measurement. For some applications, especially those relying on self-consciousness in asserting control and attention, such as distance learning and self-study, it is important to understand if a subject really focus on the participating activity.

### SUMMARY OF THE INVENTION

It is an objective of the present invention to provide a system and method that use multi-modal signals, including but not limited to EEG, photoplethysmography (PPG), inertial measurements, image, audio, etc., to recognize participating activities and compute a subject's attention level. The subject to system can be, without limitation, a child, a SEN student, a remote worker, or any person who needs attention therapy.

In one embodiment of the present invention, fusion of these multi-modal signals is performed with machine learning (ML) techniques for both activity recognition and attention computation. The system records the multi-modal signal data collected; and uses one or more of signal processing, ML, ML-based object detection, text recognition, and natural language processing (NLP), and other deep learning techniques to analyze multi-modal signal data. Further, the system may use different combinations of the multi-modal signal data to determine the activity the subject is participating in and the attention level the subject is paying on the participating activity.

The various embodiments of the present invention may be implemented by an attention level computation device particularly in distance learning, self-studying and training settings, and also meditation. Wearable devices in accordance with the present invention can be worn on a subject under attention monitoring. The multi-modal signals collected from the device being worn on the subject are sent to a processing device, such as smartphone, tablet computer, personal computer, or a remote server, for activity recognition, data and attention analysis.

The wearable device solves the problem of attention monitoring when no other people is in the presence with the subject during an attention monitoring session. The activity recognition functionality in accordance to one embodiment of the present invention solves the problem that when the subject is alone and conventional attention monitoring may only be able measure the attention level but may not be able to ensure the subject's participating activity is the intended activity (i.e., self-studying on the intended subject matter but not others or playing videogame).

In accordance with another embodiment, portions or whole of the multi-modal data collected and recorded by the system is shared with one or more certain authorized third parties including but not limited to, peers, educators, trainers, supervisors, parents, and therapists. The third parties may then review the recorded multi-model data of a plurality of subjects in designing guidance and curriculum, or providing medical diagnosis to the subject as appropriate. The collected multi-model data may also be used to facilitate attention competitions among multiple subjects. These attention competitions may be based on the same or similar participating activities and incentive rewards for subjects achieving desired attention levels.

### BRIEF DESCRIPTION OF THE DRAWINGS

Embodiments of the invention are described in more details hereinafter with reference to the drawings, in which:

FIG. 1 depicts a schematic diagram illustrating an exemplary implementation of a system for recognizing a subject's participating activities and computing the subject's attention level in accordance to one embodiment of the present invention; and

FIG. 2 depicts a process and data flow diagram of the system for recognizing a subject's participating activities and computing the subject's attention level in accordance to one embodiment of the present invention.

### DETAILED DESCRIPTION

In the following description, systems and methods for recognizing a subject's participating activities and computing the subject's attention level and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

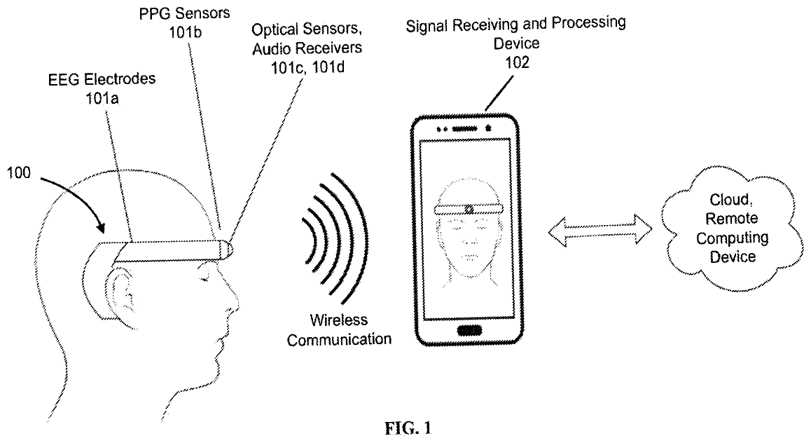

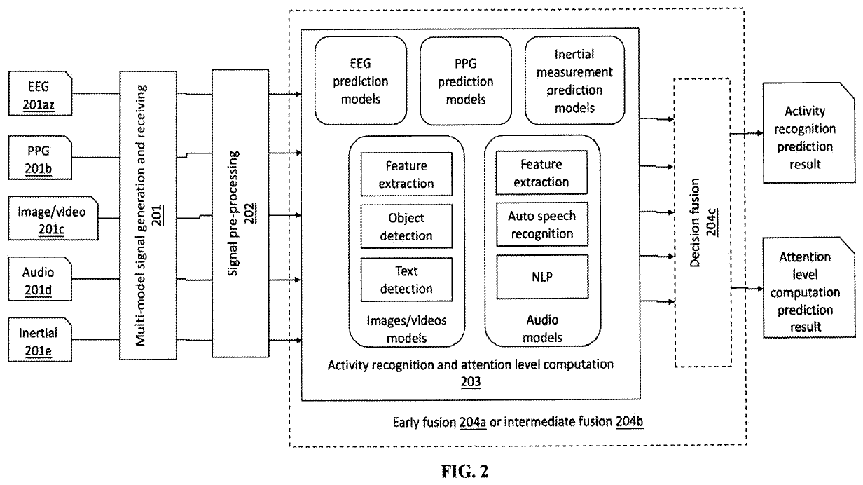

In accordance to various embodiments of the present invention, provided is a system and a method that use multi-modal signals for both activity recognition and attention computation. Referring to FIGS. 1 and 2, the system 100 comprises one or more multi-model signal sensors; and a signal receiving and processing device 102. The multi-model signal sensors comprise one or more of, EEG electrodes 101a, PPG sensors 101b, optical sensors 101c for capturing images and videos, and audio receivers 101d for capturing audios. If an implementation of an embodiment has sufficient computing resources, the signal receiving and processing device 102 is a component within the system 100, and as such no other external equipment is required. Generally, the signal receiving and processing device 102 can be a processing server, a personal computer, a mobile computing device such as a laptop, a tablet computer, or a smartphone, or some other computing device with sufficient computing power.

Although FIG. 1 shows that the system 100 is implemented in the physical form of a headband, an ordinarily skilled person in the art can easily realize the embodiments of the present invention in other physical forms, such as a clip attachable to clothing, spectacles, wristband, armband, and other wearables.

The multi-modal signal types comprise, but not limited to, EEG (201a), PPG (201b), images/videos (201c), and audio (201d), and inertial measurements (201e) as shown in FIG. 2. For each type of the multi-modal signals under its respective technology, such as EEG signals, it can be composed of signals measured from more than one signal sensors. For example, the PPG signals may be measured from one or more PPG sensors. Each of the multi-modal signal types can be used alone or together with other signal types to identify the activity types and compute the attention levels.

The measured multi-modal signals are transmitted to the signal receiving and processing device 102 via wireless communication of technologies and protocols including, but not limited to, Bluetooth, Wi-Fi, infrared, and audio; or wired communication of technologies and protocols including, but not limited to, USB.

After one or more of the multi-modal signals and/or combinations thereof are generated and received (201), each of the measured signals is pre-processed (202) by noise filtering, weak signal segments having amplitudes below a minimum signal amplitude threshold and intermittent signal segments having continuous active durations shorter than a minimum signal active duration threshold may be ignored or discarded. Then, the pre-processed multi-modal signals are used for activity recognition and attention level computation. There are different pre-processing methods for different types of the multi-modal signals. For example, when an EEG electrode is not in sufficient contact with the skin, the measured signal quality can deteriorate. Some signals, such as EEG signals, PPG signals, etc., are vulnerable to the ambient environmental interferences, such as common AC electrical frequency interference. For AC electrical frequency interference, use of notch filters may reduce the impact of the AC power source. For PPG signals (201b) and images/videos (201c), effect of physical vibrations on the sensors is substantial. To address this problem, an inertial measurement unit (IMU) is included in the system. When the inertial measurements (201e) obtained by the IMU indicate a change of the subject's movement beyond a maximum change of movement threshold, the corresponding segments of PPG signal (201b) and images/videos (201c) received are ignored or discarded, and/or their measurements are halted. If the system does not include an IMU, signal characteristics of the PPG signals (201b) and images/videos (201c) are used in signal quality calculation to determine if they are useful. The interference on audio signals (201d) and background ambient noise can be reduced by simple filtering, or by a ML technique.

In accordance to one embodiment, each signal type in the multi-model signals is used differently by the activity recognition module and the attention level computation module in recognizing and identifying activities and computing attention level respectively (203). Each signal type in the multi-model signals can be used alone or in combinations of one or more of the other types in optimizing the results of the activity recognition and identification and attention level computation.

It should be understood that the composition of the multi-model signal types as described in the present disclosure is not of limiting and serves only to illustrate the inventive concepts of the embodiments of the present invention. An ordinarily skilled person in the art shall appreciate other embodiments that use one or more other types of signals and/or combinations thereof are readily realizable without undue experimentation or deviation from the spirit of the present invention.

### EEG Signal

EEG signals generated and received through the one or more EEG electrodes are transmitted to the signal receiving and processing device 102 for activity recognition and attention level computation. The received EEG signals are then converted to a brainwave plot for activity recognition and attention level computation.

### Activity Recognition:

Based on current state of technology, brainwave plots can be used to identify a limited number of activity types. However, brainwave plots are not suitable for identifying dynamic activities. For other types of activities, the EEG activity recognition in accordance with an embodiment of the present invention identifies a representative pattern of the brainwave plot and employs one of a trained neural network, such as a convolution neural network (CNN), a Support Vector Machine (SVM), a Random Forest classifier, and a ML prediction model in processing the brainwave plot's representative pattern of an EEG signal to predict the type of activity.

### Attention Level Computation:

The EEG attention level computation employs a ML prediction model based on the frequency analysis on brainwave plot's representative pattern of the received EEG signals to predict the subject's attention level. The EEG attention level computation can be combined with one or more of attention level computation of the other multi-modal signal types to more accurately measure the subject's attention levels.

### PPG Signal

PPG signals generated and received through the one or more PPG sensors, such as pulse oximeters, are transmitted to the signal receiving and processing device 102 for activity recognition and attention level computation.

### Activity Recognition:

In general, a PPG signal contains information of heart rate and respiration. A motion-corrupted PPG, on the other hand, contains not only the heart rate and respiration information, but also the motion artifact information. In accordance with one embodiment, a received PPG sign is first detected for motion artifact data for extraction. The PPG activity recognition then employs a ML prediction model based on a Random Forest classifier, a SVM, or a neural network trained end-to-end to predict activity types from the extracted motion artifact data in PPG signals. Although activity recognition based on motion artifact information is not as accurate as that based on motion information measured by the IMU, especially for sports activities; the PPG signals can be used for activity recognition independent of other signal types and activity recognition methods.

### Attention Level Computation:

A typical PPG sensor may include an artificial light source, such as a LED, for illuminating the subject's skin, and a photodiode sensor for measuring the change in the reflected light intensity caused by the subject's blood flow. The visual representation of a PPG signal looks similar to an arterial blood pressure (ABP) waveform. The subject's pulse and the heart rate variability (HRV) can be measured as well. The pulse frequency and changes of the heart rate are related to the subject's attention level, and studies revealed that the HRV is significantly reduced during sustained attention. As such, one ML prediction model of the PPG attention level computation comprises analyzing the subject's pulse frequency and HRV.

660 nm red light and 940 nm near-infrared light are used in measuring the change of hemoglobin in the subject's blood flow. The photon absorption rate measured reflects the oxygen content in the blood. Blood oxygen saturation (SpO2) is an indicator of the normal functioning of the body and it affects many physical conditions related to attention level.

Generally, for a healthy person, the normal blood oxygen level is about 97-98%, and low blood oxygen level is approximately 90%. When the blood oxygen level reaches 90% or below, it means the subject is entering a state of insufficient attention. Under normal circumstances, a below-90% low blood oxygen level rarely occurs, and sustained low blood oxygen levels may also be related to health problems. Thus, it is not so accurate to measure attention level with only blood oxygen level.

In accordance to one embodiment, a functional near-infrared spectroscopy (fNIRS) with a light source of red light at 660 nm and another of a near-infrared light at 940 nm is used to measure blood oxygen saturation. The fNIRS is an optical neuroimaging device developed based on the principles of neurovascular coupling and spectroscopy. The increase in neural activity leads to an increase in oxygen metabolism, which is necessary to meet the energy requirements of neuronal tissue (neurometabolic coupling). In neuronal oxygen metabolism, oxygen is consumed to produce energy, resulting in a decrease in the oxygen concentration of hemoglobin. The fNIRS is to measure the changes in the oxygen concentration of neurons in the gray matter of the brain. In the fNIRS, the distance between the emission and the photodiode is several times longer than that of a pulse oximeter, which can be 2-3 cm, or even 6-7 cm. In addition, the fNIRS is similar to the multi-channel EEG electrodes in that a plurality of optopolar caps is commonly used to collect multi-channel signal data. Depending on the positions of the optopolar caps and the intensity of the measured signals, the signals can be processed and visualized in similar fashion as the functional magnetic resonance imaging. Therefore, under another ML prediction model, the PPG attention level computation uses fNIRS-measured signals like brainwave plot data in predicting the subject's attention level.

### Image/Video Signal

Image/video signal data contains a sequence of images generated and received with defined continuous shooting frequency and duration through the one or more optical sensors and are transmitted to the signal receiving and processing device 102 for activity recognition and attention level computation. With the received image/video signal data, object detection is performed on the images. In one embodiment, the object detection is a feature-based object detection, wherein the features may include one or more of color, histogram, frequency, edges, corners, lines, shapes, and contours; and feature descriptors such as Histogram of Oriented Gradient (HoG), Scale-Invariant Feature Transform (SIFT), and Speeded-up Robust Features (SURF). In another embodiment, the object detection is an attribute-based objection detection, wherein the attribute may include one or more of object class, object size, object orientation and position, text font size, text font type, text font style, and text orientation and position. In various embodiments, the object detection further comprises a text detection for specifically detecting textual elements in the images.

### Activity Recognition:

The detected objects and the combination thereof reflect the condition of the surrounding environment of the subject and the type of activity that the subject is performing. For example, when the subject is reading books in a solitary setting, the detected objects should include book covers, pictures, and texts. The subject's surrounding environment and the detected objects are used in the prediction model of the image/video activity recognition.

Depending on the frequency of continuous shooting of the images, within a certain frequency range, the detected objects should rarely change from one image to another. However, when the shooting frequency is too slow or the shooting duration is too short, there may not have enough images generated for sufficient number of objects being detected for reasonably accurate classification of the activity in the image/video activity recognition. During the objection detection, the detected objects can be numerous in types, which can confuse the activity classification as to what activity the subject is actually performing, especially in situations where there are other people in the subject's surrounding environment, the subject is interacting with another person, or the subject is performing multiple activities simultaneously. In addition, the object detection is prone to errors, which can be defined through metrics such as Precision and Recall, Intersection over Union (IOU), Mean Average Precision (mAP), etc. As such, the object detection employs an object detection confidence system. In one embodiment, only the detected objects with degrees of confidence higher than a confidence threshold (i.e., 30%), the detected objects are validated to proceed to activity classification in the image/video activity recognition. In an alternative embodiment, all detected objects are sorted by their degrees of confidence and the X number of detected objects with the highest degrees of confidence are validated to proceed to activity classification in the image/video activity recognition.

In various embodiments, the object detection may be implemented based on Tensorflow, Pytorch, or other similar ML frameworks built on trained neural networks, such one being a CNN. The results obtained from the object detection and confidence processing comprising the detected objects, the object type of each of the detected objects, quantity of detected objects of each of the object types, distribution of the object types, temporal information of the detected objects, and relationships among the detected objects. Examples of relationships among the detected objects include the relative positions of related objects such as a hand and a pen. The results obtained from the object detection and confidence processing are used in a prediction model based on a Random Forest classifier, a SVM, a neural network trained end-to-end, or other ML-based classifier to recognize the activity.

### Attention Level Computation:

When the subject is performing a static activity, and when the subject is paying little attention to the activity at hands, the subject's body tends to move more frequently. As the optical sensor being wore on the subject, relatively more blurred images are generated, or that there are more changes in the scene of the subject's surrounding environment and/or the detected objects. Thus, from the analysis of the image characteristics, the detected objects, and the frame-to-frame changes thereof, the subject's attention level can be deduced.

For dynamic activities having simple and repetitive movements, an image scene model for each type of such dynamic activities can be built by a ML-based algorithm using training datasets collected from a sample of individuals performing the type of dynamic activity with high level of attention. With the dynamic activity image scene model established, the image/video signal data generated and received in run-time are compared with the dynamic activity image scene model. The smaller the difference is, and the more the repetitions are in the run-time image/video signal data, the higher the probability that the subject is performing the dynamic activity with is high attention.

For dynamic activities having little or no repetitive movements, there are not enough data to build a model, hence it is impossible to compare the changes in the images to compute attention level. For these types of dynamic activities, other multi-modal signal types are to be relied upon for attention level computation.

### Audio Signal

Audio signals generated and received through the one or more audio receivers are transmitted to the signal receiving and processing device 102 for activity recognition and attention level computation. When the activity involves sound in the ambience of the subject such as an in-person open conversation or music performance, an audio receiver, such as a microphone, suffices in recording such audio signals. However, when the activity involves sound output from closed audio source, such as an earphone, the audio signals are to be collected directly from the audio source.

### Activity Recognition:

Features of the audio signals received are extracted through the use of spectrum analysis based on one or more of mel frequency cepstral coefficient (MFCC), Gammatone cepstral coefficient (GCC), and linear prediction cepstral coefficients (LPCC). The extracted features are then used for identifying the activity type.

For audio signals received that contain speech contents, such as conversations, the speech is first recognized and converted into texts by an automatic speech recognition tool such as Kaldi. In one embodiment, the texts are analyzed by a natural language processing (NLP) tool, such as RASA and DialogFlow, for the context, intents, and entities of the speech contents, and in turn for matching the audio models of different types of activities. In another embodiment, the texts are analyzed by one of Large-Language models (i.e., ChatGPT™) and Transformer models.

There are certain situations that have little conversation or only one-way dialogue in the audio contents. For instance, in a distance learning class, teachers are speaking while students are listening, voice are mainly uttered from the teacher. Under such situation, the audio signals received from the student subject's audio receiver mainly contain the teacher's voice and background ambient noise, or just ambience sound. Nevertheless, an audio model can still be built for recognizing such types of activities based on extracted features of audio signals. Similarly, audio models can be built for other types of activities.

### Attention Level Computation:

Similar to the audio activity recognition, with the use of the speech recognition and NLP tools in one embodiment, the subject's attention level can be determined by analyzing the degree of relevance of the subject's dialogue to the context of the speech contents in the audio signals; and the subject's response speed. The subject's attention level is then proportional with the degree of relevance of the subject's dialogue and response speed. In another embodiment, the speech in audio signal is analyzed by one of Large-Language models and Transformer models. For activities in which the subject seldom utters or only ambience sound is present, the attention level can still be computed by the audio models specifically built for such types of activities based on extracted features, such as silence time, speaker identification, and utter's spectrum, of audio signals.

### Inertial Measurement Signal

Multi-axis inertial measurement signals generated and received through the IMU are transmitted to the signal receiving and processing device 102 for activity recognition and attention level computation.

### Activity Recognition:

The use of IMU's to identify human activities has been developed for a long time. Many commercially available products, such as the Fitbit™, Xiaomi™ bracelet, Apple iWatch™, and a number of smartphone apps use various types of IMU or motion sensors to detect and measure the wearer's movements and in turn recognize the types of activity and extract statistical information such as calories consumption and exercise time. In one embodiment, the inertial measurement activity recognition employs one such prediction model. However, in the recognition of fine-grained actions, the IMU can only distinguish limited types of activities.

### Attention Level Computation:

Under many non-sports static activities, a relatively low inertial measurement signal amplitude usually is associated with the subject's state of high attention. On the contrary, under dynamic activities such as competitive sport activities, the inertial measurement signal as generated and received through the IMU may simply reflect the conditions and movements in the performance of the dynamic activities. In one embodiment, a movement model for each type of such dynamic activities is learned by a ML-based algorithm using training datasets collected from a sample of individuals performing the type of dynamic activity with high level of attention. With the dynamic activity movement model established, the inertial measurement signal data generated and received in run-time are compared with the dynamic activity movement model. The smaller the difference is, and the more the repetitions are in the run-time inertial measurement signal data, the higher the probability that the subject is performing the dynamic activity with high attention level.

### Alternative Embodiment of Activity Recognition and Attention Level Computation

In accordance with an alternative embodiment of the present invention, activity recognition and attention level computation are achieved using a K-means cluster algorithm and the Generalized Singular Value Decomposition (GSVD) technique. In this alternative embodiment, the brainwave plot data of the EEG signals received and inertial measurement signal data received are input to the K-means cluster algorithm for classification. The input data are divided into L1, L2 and L∞ standard categories. Because the characteristics of L1, L2 and L∞ standard categories are different from each other, they can be used to identify the different types of activities; by which the subject's activity type and attention level can be estimated.

Under the GSVD technique, two data of the same type can be made as GSVD, and the diagonal elements of their diagonal matrix can be used as features. Then, the corresponding feature vectors are classified according to the standard criteria of L1, L2 and L∞. The decision boundary of L1 and L∞ is segmental linear, hence the decision range of L1 and L∞ is non-convex. On the other hand, the decision boundary of L2 is linear, hence the decision range of L2 is convex. The average value K is then used to estimate the subject's activity type. For example, when the K-mean value is closer to point A, the subject is performing the activity of A, and so on to points B and C. More specifically, the K-means of the L2 standard is calculated by a mixed-integer quadratic equation, and the K-means of the L1 and L∞ standard is calculated by a linear equation of mixed integer.

### Models Fusion

In various embodiments, each type of the multi-modal signal types is used in its respective activity recognition and attention level computation independent of the other signal types. In these embodiments, each of the activity recognitions and each of the attention level computations has its own prediction model or ML-based algorithm. In other embodiments, multiple signal types are used in combination to produce more accurate prediction results in the activity recognition and attention level computation. And for some of the signal types, such as image/video and audio, the prediction models can be combined to form fused prediction models for processing the different signal types together.

In one embodiment, the prediction models in the activity recognitions of all of multi-modal signal types are fused and the prediction results in the attention level computations of all of multi-modal signal types are also fused under an early fusion strategy (204a). Under the early fusion strategy (204a), the pre-processed output and/or extracted features of the signal data of all of multi-modal signal types are merged before inputting to the unified prediction models (one for activity recognition and one for attention level computation).

In another embodiment, the prediction models in the activity recognitions of all of multi-modal signal types are fused and the prediction results in the attention level computations of all of multi-modal signal types are also fused under an intermediate fusion strategy (204b). Under the intermediate fusion strategy (204b), some of the multi-modal signal types have their own activity recognition prediction models and attention level computation prediction models, and their prediction results are merged with the pre-processed output and/or extracted features of the signal data of some of the other multi-modal signal types as input to further activity recognition prediction models and attention level computation prediction models.

In yet another embodiment, the prediction models in the activity recognitions of all of multi-modal signal types are fused and the prediction results in the attention level computations of all of multi-modal signal types are also fused under a late fusion (or decision fusion) strategy (204c). Under the decision fusion strategy (204c), each of the multi-modal signal types has its own activity recognition prediction model and attention level computation prediction model. The individual prediction results on the multi-modal signals of all types are aggregated in an ensemble learning, which may include one or more of majority voting, averaging, and weighted voting.

The functional units and modules of the systems for recognizing participating activities and computing a subject's attention level in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A method for recognizing a participating activity and computing an attention level of a subject from multi-model signals, comprising:
receiving the multi-model signals comprising one or more of an electroencephalogram (EEG) signal generated and received through one or more EEG electrodes, a photoplethysmography (PPG) signal generated and received through one or more PPG sensors, an image/video signal generated and received through an optical sensor, an audio signal generated and received through an audio receiver, and an inertial measurement signal generated and received through an inertial measurement unit (IMU);
executing, by a signal receiving and processing device, an activity recognition to predict an activity type of a participating activity being performed by the subject using one or more of the multi-model signals; and
executing, by the signal receiving and processing device, an attention level computation to predict the subject's attention level in performing the participating activity using one or more of the multi-model signals.

2. The method of claim 1, further comprising:
pre-processing the multi-model signals before the executions of the activity recognition and the attention level computation, the pre-processing comprising:
discarding one or more signal segments in the multi-model signals having amplitudes below a minimum signal amplitude threshold or having continuous active durations shorter than a minimum signal active duration threshold;
reducing AC electrical frequency interferences in the EEG signal and the PPG signal using one or more notch filters;
discarding one or more of the PPG signal segments in the multi-model signals generated and received when physical movement of the PPG sensor exceeds a maximum change of movement threshold;
discarding one or more of the image/video signal segments in the multi-model signals generated and received when physical movement on the optical sensor exceeds a maximum change of movement threshold; and
filtering out background ambient noise of the audio signal.

3. The method of claim 1, wherein the activity recognition comprises an EEG activity recognition and the attention level computation comprises an EEG attention level computation;
wherein the EEG activity recognition comprising:
converting the EEG signal to a brainwave plot;
identifying a representative pattern of the brainwave plot; and
employing one of a trained neural network, a Support Vector Machine (SVM), a Random Forest classifier, and a ML prediction model to predict the activity type of the participating activity from the representative pattern of the brainwave plot; and
wherein the EEG attention level computation comprising:
employing a ML prediction model based on frequency analysis on the representative pattern of the brainwave plot to predict the attention level.

4. The method of claim 1, wherein the activity recognition comprises an PPG activity recognition and the attention level computation comprises an PPG attention level computation;
wherein the PPG activity recognition comprising:
extracting motion artifact information from the PPG signal;
employing a ML prediction model to predict the activity type of the participating activity from the motion artifact information; and
wherein the PPG attention level computation comprising:
employing a first ML prediction model based on pulse frequency and heart rate variability analysis to predict the attention level from the PPG signal generated and received through only a single channel of the PPG sensors; or
employing a second ML prediction model based on functional near-infrared spectroscopy (fNIRS) analysis to predict the attention level from the PPG signal generated and received through multiple channels of the PPG sensors.

5. The method of claim 1, wherein the activity recognition comprises an image/video activity recognition and the attention level computation comprises an image/video attention level computation;
wherein the image/video activity recognition comprising:
performing one of feature-based object detection, attribute-based object detection, and ML-based objection detection using a trained neural network to detect objects in the image/video signal;
selecting the detected objects using an object detection confidence system; and
employing a ML prediction model to predict the activity type of the participating activity from the selected-detected objects;
wherein the image/video attention level computation comprising:
for static activity type, employing a ML prediction model based on analysis of image characteristics, the selected-detected objects, and frame-to-frame changes to predict the attention level from the image/video signal; and
for dynamic activity type, comparing the image/video signal to an image scene model for the activity type of the participating activity to estimate the attention level.

6. The method of claim 1, wherein the activity recognition comprises an audio activity recognition and the attention level computation comprises an audio attention level computation;
wherein the audio activity recognition comprising:
extracting features of the audio signal using a spectrum analysis method;
for audio signal containing speech contents:
recognizing and converting the speech contents into texts;
analysing the texts using one of a natural language processing (NLP) tool, a Large-Language model, and a Transformer model for context, intents, and entities of the speech contents; and
employing a ML prediction model to predict the activity type of the participating activity from the context, intents, and entities of the speech contents; and
for audio signal containing no speech content:
employing a ML prediction model to predict the activity type of the participating activity from the extracted features of the audio signal;
wherein the audio attention level computation comprising:
for audio signal containing speech contents:
determining a degree of relevance of the subject's dialogue in the speech contents;
determining a response speed of the subject in the speech contents; and
employing a ML prediction model to predict the attention level from the e degree of relevance of the subject's dialogue and the response speed of the subject; and
for audio signal containing no speech content:
employing a ML prediction model to predict the attention level from the extracted features of the audio signal.

7. The method of claim 1, wherein the activity recognition comprises an inertial measurement activity recognition and the attention level computation comprises an inertial measurement attention level computation;
wherein the inertial measurement activity recognition comprising:
employing a ML prediction model to predict the activity type of the participating activity from the inertial measurement signal;
wherein the inertial measurement attention level computation comprising:
comparing the inertial measurement signal to a movement model for the activity type of the participating activity to estimate the attention level.

8. The method of claim 1, further comprising:
fusing all prediction results of activity recognitions from the multi-model signals under decision fusion strategy; and
fusing all prediction results of attention level computations from the multi-model signals under decision fusion strategy.
